(19) United States
(12) Patent Application Publication
(10) Pub. No.: US 2008/0172777 A1
(43) Pub. Date: Jul. 24, 2008

(54) Swimming goggles

(76) Inventor: Herman Chiang, Chung-Ho City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/705,015

(22) Filed: Feb. 12, 2007

(30) Foreign Application Priority Data
Jan. 18, 2007 (TW) 096201022

(51) Int. Cl. A61F 9/02

(52) U.S. Cl. 2/428

(57) ABSTRACT

Swimming goggles include a left frame, a right frame, a connecting portion between the left frame and the right frame, lenses, strap elements and a head strap. The left frame, the right frames and the connecting portion are made of soft material. The lenses and the strap elements are made of hard material. The lenses respectively form positioning portions thereon. The strap elements form embedding portions thereon for engaging with the positioning portions. The lenses and the strap elements are preliminarily assembled. Then the left frame, the right frame and the connecting portion are integrally shaped to envelop outer rims of the lenses. The strap elements are partially exposed outside for guiding the head strap. The soft left and right frames, and the connecting portion provide comfortable contact for users. The connecting portion is so flexible that fits to various users of different face profiles and prevent from water-leakage in use.

### BACKGROUND OF THE INVENTION

1. Field of the Invention

The present invention relates to swimming goggles, and particularly to swimming goggles which have soft frames and hard lenses and which provide comfortable touch for users and are adjusted conveniently in use.

2. Related Art

In general, swimming goggles are divided into two types. The first type includes a left frame and a right frame, which are separate from each other and are connected by a connecting member. The second type includes a left frame and a right frame, which are integrated with a connecting member together. Regarding the second type, material of the left frame and the right frame is required to be hard enough for reliably fixing lenses on the left frame and the right frame. Accordingly, material of pads is confined, and correspondingly, pads on the left frame and the right frame are relatively rigid, making users feel uncomfortable.

Furthermore, the connecting member may be inflexible due to the hard material, so it cannot fit various users with different face profiles. The left frame and the right frame may be too rigid to fit close to users' faces, therefore taking risk of water seepage.

### SUMMARY OF THE INVENTION

To overcome the deficiencies, an object of the present invention is to provide swimming goggles which provide comfortable feeling for users and prevent from water leakage effectively.

The swimming goggles comprise a left franks a right frame, a connecting portion between the left frame and the right frame, lenses and strap elements. The left frame, the right frames and the connecting portion are made of soft material. The lenses and the strap elements are made of hard material. The lenses respectively form positioning portions thereon. The strap elements form embedding portions thereon for engaging with the positioning portions. The lenses and the strap elements are preliminarily assembled. Thereafter, the left frame, the right frame and the connecting portion are formed in the manner of integrally shaping and envelop outer rims of the lenses. Parts of the strap elements are exposed outside for guiding a head strap.

Each strap element includes a body, a couple of legs extending from the body, and a couple of branches. The embedding portions are respectively formed on the legs. The branches extend from the body with inclined angle relative to the body and in a reverse direction relative to the legs. The branches are spaced a distance from each other. Axis holes are respectively defined near outward ends of the branches and correspond to each other. An axis lever extends through the axis holes.

The embedding portions are protrusions which are integrally formed on the legs. The positioning portions are through holes. The protrusions cooperate with the through holes for reliably assembling the lenses from inside to outside.

An L-shaped clamp is provided on the body and between the legs for abutting the lens toward inside.

Support shafts are respectively formed on the branches and near the axis holes. Pressing plates are pivoted to the support shafts. Each pressing plate has a stop arm on an inward side thereof. The stop arm may abut stop grooves of the head strap in a single direction. When the pressing plates are drawn in such a way that the stop arm disengages from the stop groove, length of the head strap is allowed to be adjusted freely.

### BRIEF DESCRIPTION OF THE DRAWINGS

FIG. 1 is an exploded view of swimming goggles of the present invention.

FIGS. 2A-2C and 3 respectively show the swimming goggles being assembled step by step.

FIG. 4 is a front view of the swimming goggles of FIG. 3.

FIG. 5 is a cross-sectional view taken along the line 5-5 in FIG. 4.

FIG. 6 is a partially enlarged view of FIG. 5.

### DESCRIPTION OF THE PREFERRED EMBODIMENTS

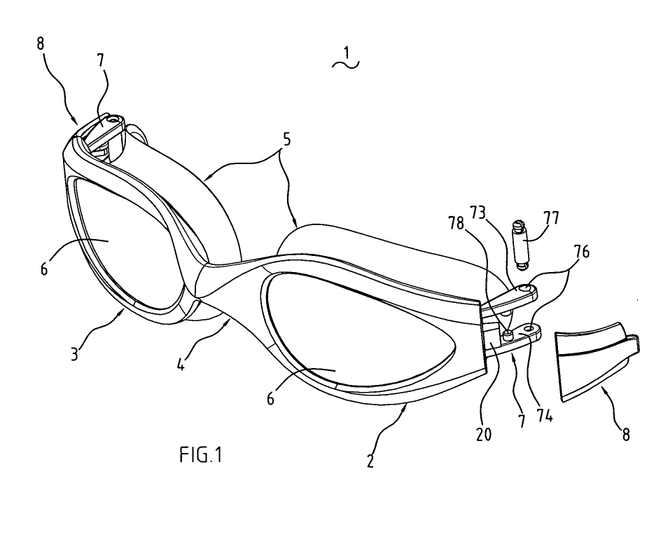

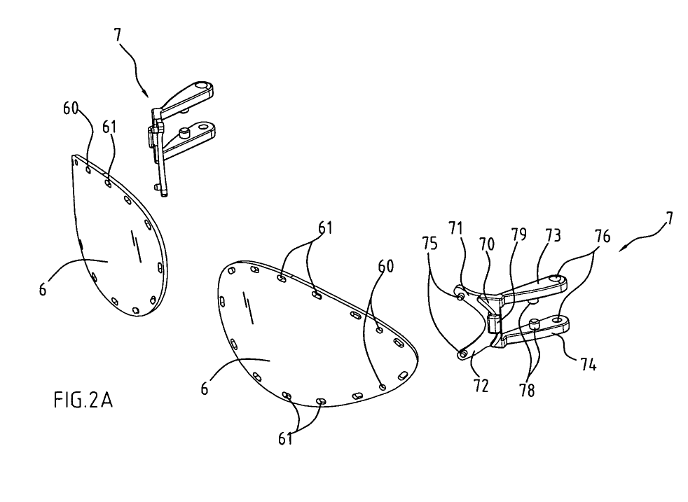

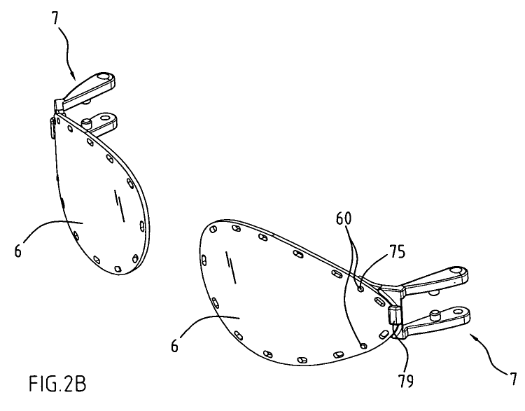

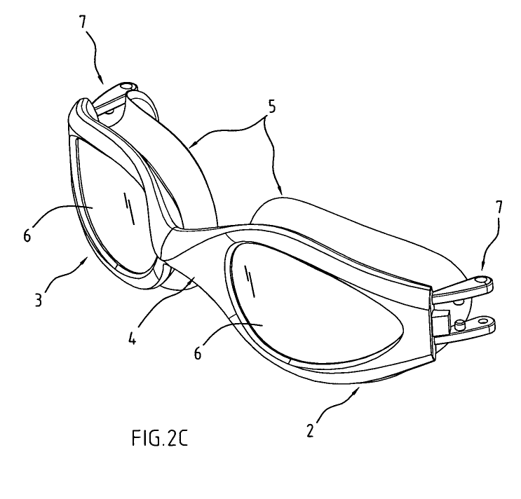

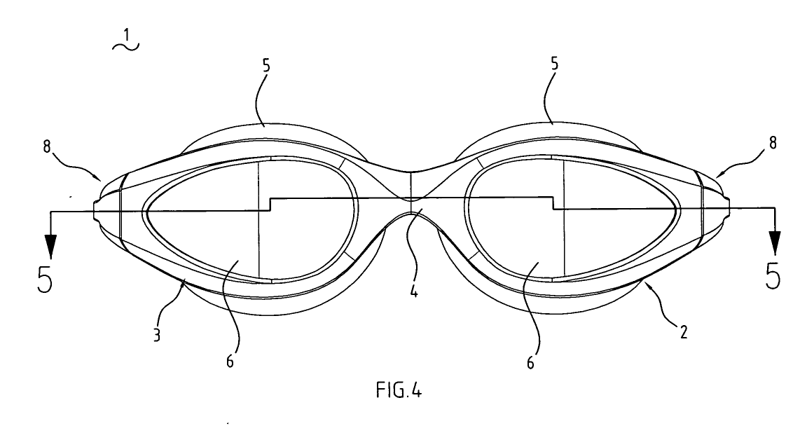

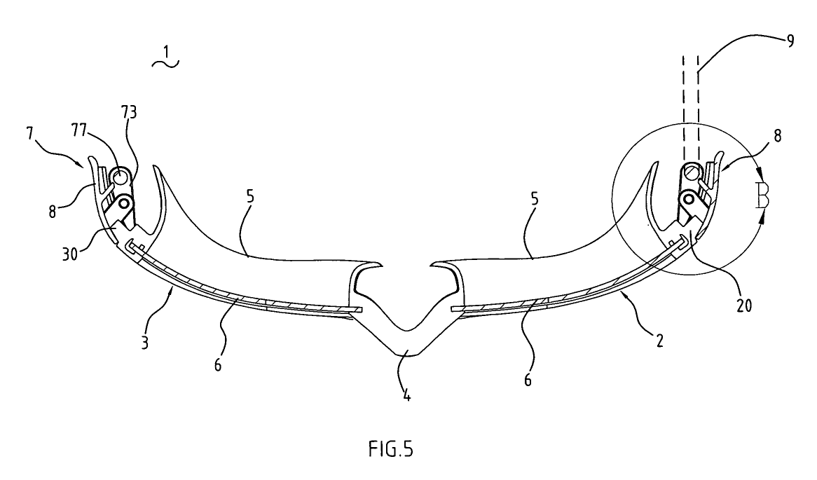

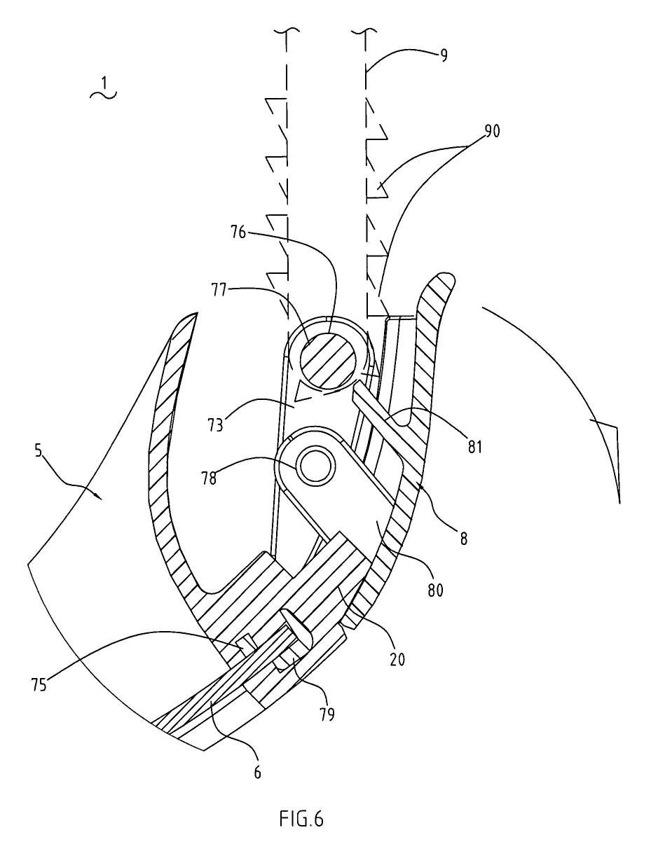

With reference to FIG. 1, swimming goggles 1 in accordance with the present invention comprise a left frame 2, a right frame 3, a connecting portion 4 between the left frame 2 and the right frames 3, pads 5, lenses 6, strap elements 7, pressing plates 8 and a head strap 9 (shown in FIGS. 5 and 6). The left frame 2, the right frame 3 and the connecting portion 4 are unitarily formed and are made of the same soft material, for example, silica gel. The lenses 6 and the strap elements 7 are made of hard material, for example, PP material. The pads 5 are integrated with the left frame 2 and the right frame 3. Further viewed from FIGS. 2A to 2C, each lens 6 respectively forms positioning portions 60 on an upper portion and a lower portion thereof and near an outward rim thereof. In this embodiment the positioning portions 60 are through holes 60. At least an injecting hole 61 is distributed about the positioning portions 60 for the purpose of plastic passing therethrough when being shaped. Each strap element 7 includes a body 70, a couple of legs 71, 72 and a couple of branches 73, 74. The legs 71, 72 are integrally extended from the body 70, and correspond to each other to be formed with an arc shape for fitting to an arcuate outward rim of the lens 6. Embedding portions 75 are formed unitarily on the legs 71, 72 for engaging with the positioning portions 60. For example, the embedding portions 75 are protrusions 75 in this embodiment. The legs 71, 72 cooperate with the through holes 60 for assembling the lenses 6 from inside to outside. The branches 73, 74 integrally extend from the body 70 with inclined angle relative to the body 70 and in a reverse direction relative to the legs 71, 72. The branches 73, 74 are spaced a distance from each other. Axis holes 76 are respectively defined near outward ends of the branches 73, 74 and correspond to each other for assembling an axis lever 77 (shown in FIG. 5) which guides the head strap 9. Support shafts 78 are respectively formed on the branches 73, 74 and near the axis holes 76 for assembling the pressing plates 8. An L-shaped clamp 79 is provided on the body 70 and between the legs 71, 72 for abutting the lens 6 toward inside. In other words, the lenses 6 and the strap elements 7 are preliminarily oriented first. As shown in FIGS. 2A and 2B, the through holes 60 of the lenses 6 are assembled to the protrusions 75 of the branches 73, 74. The lenses 6 are respectively assembled from inside to outside. The clamps 79 abut against the lenses 6 inwardly. Further referring to FIG. 6, the lenses 6 are preliminarily positioned reliably. Then, the lenses 6 are put in a shaping mold, and are combined with the left frame 2, the right frame 3, the connecting portion 4 and the pads 5 in the way of integrally shaping, as illustrated in FIG. 2C. The integrally shaped plastic envelops the outer rims of the lenses 6, while parts of strap elements 7 are exposed outside for guiding the head strap 9. The left frame 2 and the right frame 3 respectively form tabs 20, 30 (shown in FIG. 4) on outward sides thereof. The tabs 20, 30 are oriented corresponding to the pressing plates 8 for providing appropriate resiliency for the pressing plates 8. The lenses 6 and the strap elements 7 are preliminarily positioned, and thus, the left frame and the right frame are not subject to the movement of the head strap 9. As a result, relatively soft material is acceptable for the left frame and the right frame to provide comfortable touch and leakproof effect.

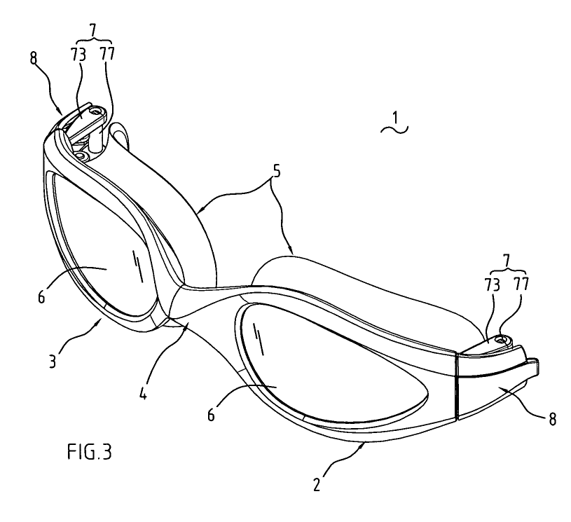

Referring to FIG. 3, the pressing plates 8 are assembled to the combination of the lenses 6, the left frame 2, the right frame 3, the connecting portion 4 and the pads 5. Also referring to FIGS. 1 and 6, each pressing plate 8 has an assembling arm 80 and a stop arm 81 on an inward side thereof. The assembling arm 80 pivots to the support shaft 78, and therefore the assembling arm 80 pivots about the support shaft 78. The stop arm 81 may abut against stop grooves 90 of the head strap 9 in a single direction. In the case where the pressing plates 8 are pressed in a direction as the arrow in FIG. 6, the stop arm 81 disengages from the stop groove 90. Meanwhile length of the head strap 9 is allowed to be adjusted freely.

Referring to FIGS. 4, 5 and 6, in normal state, the stop arms 81 of the pressing plates 8 abut against the stop grooves 90 of the head strap 9, retaining the head strap 9 fixedly. For the purpose of loosening the head strap 9, as shown in FIG. 6, the pressing plates 8 are drawn outwardly (in a direction as the arrow in FIG. 6). The stop arms 81 disengage from the stop grooves 90 of the head strap 9, and thereafter the head strap 9 is permitted to be adjusted. At this time the tabs 20, 30 are pressed and preserve energy. When the pressing plates 8 are drawn, the tabs 20, 30 drive the pressing plates 8 to return to original positions owing to preserved energy. The pressing plates 8 facilitate to adjust length of the head strap 9 conveniently.

It is understood that the invention may be embodied in other forms without departing from the spirit thereof. Thus, the present examples and embodiments are to be considered in all respects as illustrative and not restrictive, and the invention is not to be limited to the details given herein.

1. Swimming goggles comprising a left frame, a right frame, a connecting portion between the left frame and the right frame, lenses, strap elements and a head strap, the left frame and the right frame being made of soft material while the lenses and the strap elements being made of hard material, the lenses respectively forming positioning portions thereon, the strap elements forming embedding portions thereon for engaging with the positioning portions, the lenses and the strap elements being preliminarily assembled, and the left frame, the right frame and the connecting portion being formed by integrally shaping to envelop outer rims of the lenses, parts of the strap elements being exposed outside for guiding the head strap.

2. The swimming goggles as claimed in claim 1, wherein each strap element includes a body, a couple of legs and a couple of branches, the embedding portions being respectively formed on the legs, the branches extending from the body with inclined angle relative to the body and in a reverse direction relative to the legs, the branches being spaced a distance from each other, axis holes being respectively defined near outward ends of the branches and corresponding to each other, an axis lever extending through the axis holes.

3. The swimming goggles as claimed in claim 2, wherein the embedding portions are protrusions which are integrally formed on the legs, and wherein the positioning portions are through holes, the protrusions cooperating with the through holes for assembling the lenses from inside to outside.

4. The swimming goggles as claimed in claim 3, wherein the legs correspond to each other and are formed with an arc shape for fitting to arcuate outward rims of the lenses.

5. The swimming goggles as claimed in claim 4, wherein at least an injecting hole is distributed about the positioning portions for the purpose of plastic passing therethrough when being shaped.

6. The swimming goggles as claimed in claim 2, wherein support shafts are respectively formed on the branches and near the axis holes, and wherein pressing plates are pivoted to the support shafts, each pressing plate including an assembling arm on an inward side thereof for pivoting to the support shafts, and a stop arm for abutting stop grooves of the head strap in a single direction, length of the head strap being allowed to be adjusted freely when the pressing plates are drawn in such a way that the stop arms disengage from the stop grooves.

7. The swimming goggles as claimed in claim 6, wherein the left frame and the right frame respectively form tabs thereon for providing resiliency, thereby driving the pressing plates to return by preserved energy when the pressing plates are pressed.

8. The swimming goggles as claimed in claim 2, wherein an L-shaped clamp is provided on the body and between the legs for abutting against the lens toward inside.

9. The swimming goggles as claimed in claim 1, wherein the left frame, the right frames and the connecting portion are unitarily formed and are made of the same soft material.

10. The swimming goggles as claimed in claim 9, wherein pads are integrated with the left frame and the right frame.
